(19) United States
(12) Patent Application Publication
(10) Pub. No.: US 2022/0380477 A1
(43) Pub. Date: Dec. 1, 2022

(54) PHARMACEUTICAL COMPOSITION COMPRISING PRION PROTEIN-SPECIFIC ANTIBODY

(71) Applicant: STEM BIO CO., LTD, Asan-si, Chungcheongnam-do (KR)

(72) Inventor: Sang Hun LEE, Yongin-si, Gyeonggi-do (KR)

(21) Appl. No.: 17/787,517

(22) PCT Filed: Jul. 8, 2020
(86) PCT No.: PCT/KR2020/008912

§ 371 Date: Jun. 20, 2022

(30) Foreign Application Priority Data
Dec. 20, 2019 (KR) 10-2019-0172531

(51) Int. Cl. C07K 16/28; A61K 39/395; A61K 31/513; A61K 31/282; A61P 35/00; A61P 1/00; A61P 35/04

(52) U.S. Cl. CPC C07K 16/2872; A61K 39/3955; A61K 31/513; A61K 31/282; A61P 35/00; A61P 1/00; A61P 35/04; A61K 2039/505

(57) ABSTRACT

Proposed are a pharmaceutical composition for cancer treatment, comprising a prion protein-specific antibody, and a pharmaceutical composition for cancer treatment, comprising a complex of a target that binds to a prion protein antigenic determinant, and one or more selected from the group consisting of an anticancer drug, a chemotherapeutic agent, a toxin, a radioisotope, and a cytotoxic enzyme.

### TECHNICAL FIELD

The present invention relates to a pharmaceutical composition comprising a prion protein-specific antibody.

### BACKGROUND ART

Colorectal cancer is the most commonly diagnosed cancer worldwide and is the third most common cause of death in recent years. The cause of colorectal cancer is sporadic, and colorectal cancer is known to be caused by genetic and environmental factors. Among environmental factors, dietary factors and lifestyle are known to play the most important role. However, colorectal cancer is known to be curable when diagnosed at the early non-metastatic stage, and the five-year survival rate is as high as 90% or more.

However, when colorectal cancer metastasizes to the lymph nodes, the five-year survival rate decreases to less than 10% since metastasis to other organs is possible. Several recent studies suggest that molecularly targeted therapy and early diagnosis are potential approaches for colorectal cancer treatment. Therefore, it can be said that inhibiting the mechanism of colorectal cancer and a corresponding new target is greatly important to effectively enhance the effect of anticancer treatment and improve the life of the patient.

Normal prion protein (PrP^{C}) is a glycoprotein commonly expressed in neurons and regulates cell differentiation, cell migration, and neural substance exchange. Recent studies have reported that prion protein promotes uncontrolled cancer cell proliferation, tumor growth and metastasis. Prion protein is presented as an effective biomarker for confirming the harmful prognosis of patients with cancer such as colon cancer, rectal cancer, adenoma, breast cancer, and neuroblastoma. Here, the biomarker refers to an indicator that can detect changes in the body using proteins, DNA, RNA, cell metabolites and the like.

In particular, studies report that the prion protein is overexpressed in resistant cancer cells, cancer stem cells, and the like. In these study reports, it is mentioned that prion protein is a favorable therapeutic target for various cancers including colorectal cancer. Hence, glycoproteins such as prion protein are not effective targets for drug therapy, but methods for effectively controlling the activity of prion protein and enhancing sensitivity to anticancer drugs are needed.

Recently, exosomes secreted from tumors contain various proteins or microRNAs that have not yet been identified. Tumor-derived exosomes are known to be directly involved in tumors or to affect the tissues of major body organs through blood in the body. As these effects, it has been reported that tumor-derived exosomes have various functions such as tumor recurrence, tumor metastasis, induction of tumor resistance to anticancer drugs, and formation of an environment for tumor cell infiltration by reducing the binding ability of vascular tissues.

### SUMMARY OF INVENTION

### Technical Problem

The present invention has been proposed to solve the above problems, and an object thereof is to provide a pharmaceutical composition for cancer treatment that enhances the sensitivity of an anticancer drug by inhibiting the activity of prion protein in the body.

### Solution to Problem

The present invention has been proposed to solve the above problems, and can provide a pharmaceutical composition for cancer treatment comprising a prion protein-specific antibody.

The pharmaceutical composition for cancer treatment of the present invention may further comprise one or more selected from the group consisting of an anticancer drug, a chemotherapeutic agent, a toxin, a radioisotope, and a cytotoxic enzyme, and the anticancer drug may be one or more selected from the group consisting of 5-fluorouracil (5FU) anticancer drug and oxalaplatin anticancer drug.

In an example, the present invention may provide a pharmaceutical composition for cancer treatment comprising a complex of a target that binds to a prion protein antigenic determinant; and one or more selected from the group consisting of an anticancer drug, a chemotherapeutic agent, a toxin, a radioisotope, and a cytotoxic enzyme.

### Advantageous Effects of Invention

The pharmaceutical composition for cancer treatment comprising a prion protein-specific antibody of the present invention can improve the anticancer therapeutic effect by improving the sensitivity of an anticancer drug.

### BRIEF DESCRIPTION OF DRAWINGS

FIGS. 1A-1C and 2 are photographs and graphs illustrating the results according to Example 1.

FIGS. 3A-3C and 4A-4C are photographs and graphs illustrating the results according to Example 2.

FIGS. 5A-5B and 6A-6B are photographs illustrating the results according to Example 3.

FIGS. 7A-7B and 8A-8B are photographs and graphs illustrating the results according to Example 4.

FIGS. 9A-9B and 10A-10B are images illustrating the results according to Example 5.

FIGS. 11A-11B to 13 are photographs and graphs illustrating the results according to Example 6.

FIGS. 14A-14B and 15 are graphs and photographs illustrating the experimental model and results according to Example 7.

FIGS. 16A-16B and 17A-17B are graphs illustrating the results according to Example 8.

FIGS. 18A-18B are graphs and photographs illustrating the experimental model and results according to Example 9.

### DESCRIPTION OF EMBODIMENTS

According to an embodiment, the present invention provides a pharmaceutical composition for cancer treatment comprising a prion protein-specific antibody.

The pharmaceutical composition for cancer treatment of the present invention may further comprise one or more selected from the group consisting of an anticancer drug, a chemotherapeutic agent, a toxin, a radioisotope, and a cytotoxic enzyme, and the anticancer drug may be one or more selected from the group consisting of 5-fluorouracil (5FU) anticancer drug and oxaliplatin anticancer drug.

In an example, the present invention may provide a pharmaceutical composition for cancer treatment comprising a complex of a target that binds to a prion protein antigenic determinant; and one or more selected from the group consisting of an anticancer drug, a chemotherapeutic agent, a toxin, a radioisotope, and a cytotoxic enzyme.

### EXAMPLES

Hereinafter, the present invention will be described in more detail with reference to Examples. However, the following Examples and Experimental Examples only illustrate the present invention, and do not limit the contents of the present invention.

### Preparation Example 1

Preparation of Plasma Samples of Colorectal Cancer Patients

Plasma samples of anonymous colorectal cancer patients were donated from the Human Resources Bank of Korea, and the donated samples contain limited information about the patients (age, description, tumor status of pathology, history of chemotherapy). The present experiment and the information on clinical samples were conducted under the approval of the ethics committee of the Human Resources Bank of Korea.

### Preparation Example 2

Method of Culturing Colorectal Cancer Cell Line with Anticancer Drug Resistance

General human colorectal cancer cell line SNU-C5/WT, human colorectal cancer cell line with 5FU anticancer drug resistance SNU-C5/5FUR, and human colorectal cancer cell line with oxaliplatin anticancer drug resistance SNU-C5/OXR were provided, and human colorectal cancer stem cells [S707; cancer stem cell (CSC)] was provided by Professor Steven M Lipkin, Department of Medicine, Cornell Medical University (New York, N.Y., USA). The SNU-C5-related cell lines were cultured in the presence of 5% CO₂ in an incubator at 37° C. using RPMI culture medium containing 10% fetal bovine serum. The colorectal cancer stem cells were cultured in the presence of 5% CO₂ in an incubator at 37° C. using DMEM/F12 medium with additives including non-essential amino acids (Thermo Fisher Scientific), sodium pyruvate (Thermo Fisher Scientific), penicillin-streptomycin (Thermo Fisher Scientific), B27 supplement (Thermo Fisher Scientific), N2 supplement (Thermo Fisher Scientific), 40 μg/ml heparin (Sigma-Aldrich), 20 ng/ml fibroblast growth factor (Thermo Fisher Scientific) and 40 ng/ml epidermal growth factor (Thermo Fisher Scientific).

### Preparation Example 3

Method of Culturing Colorectal Cancer Cell Line with Anticancer Drug Resistance for Spheroid Formation

The general colorectal cancer cell line (SNU-C5/WT), anticancer drug-resistant colorectal cancer cell lines (SNU-C5/5FUR, SNU-C5/OXR), and colorectal cancer stem cell line were subjected to cell suspension culture using 6-well ultra-low cluster plates for spheroid formation. The culture was performed using a growth medium (see Preparation Example 2) and then under conditions of 37° C. and 5% CO₂.

Spheroids of colorectal cancer cell lines were formed on day 7 after culture. Spheroids of each cell line were identified and measured using an optical microscope.

### Preparation Example 4

Hypoxic Culture Technique of Anticancer Drug-Resistant Cells

The 5FU anticancer drug-resistant cell line (SNU-C5/5FUR) was exposed to a hypoxic environment of 2% 02, 5% CO₂, and N₂ balance in a hypoxic culture chamber while being cultured in a growth medium (see Preparation Example 2), and cultured at 37° C. for 24 hours. The presence or absence of hypoxic culture was indicated by dividing the environment into the normal oxygen environment (indicated by N) and the hypoxic environment (indicated by H) in the drawing.

### Preparation Example 5

Exosome Extraction Technique

The 5FU anticancer drug-resistant cell line (SNU-C5/5FUR) was cultured in a hypoxic environment for 48 hours, then the culture medium was collected and centrifuged at 400 g for 10 minutes to remove cells and other debris, and a 0.45 μm syringe filter was used to prepare a culture medium. To facilitate the extraction of exosomes from the culture medium, the culture medium was concentrated through centrifugation at 2000 g for 20 minutes using 100 kDa MWCO ultrafiltration devices. From the concentrated culture medium, exosomes were extracted using ExoLutE® conditioned medium Exosome isolation kit. Solutions A, B and C were added to 8 ml culture medium, the mixture was stirred for 10 minutes for reaction, and then centrifugation was performed at 3000 g for 15 minutes. The pellets were dissolved in solution R, and then the crude extracellular vesicles were harvested in a tube through centrifugation at 700 g in column L for 5 minutes. Next, solutions B and C were added to the crude extracellular vesicles, the mixture was stirred for 15 minutes for reaction, pellets were obtained through centrifugation at 3000 g for 5 minutes and dissolved in solution R, and then purified exosomes were obtained through centrifugation at 300 g in column R for 5 minutes.

### Preparation Example 6

Western Blot Analysis

The proteins were extracted from the 5FU anticancer drug-resistant cell line (SNU-C5/5FUR)—derived exosomes, and the cellular proteins (20 μg protein) were subjected to sodium dodecyl sulfate polyacrylamide gel electrophoresis and blotted on a nitrocellulose membrane. Next, the membrane was washed with TBS-T [10 mM Tris-HCl (pH 7.6), 150 mM NaCl, and 0.05% Tween-20], then blocked with 5% skim milk for 1 hour, and cultured with primary antibodies (PrP^{C}, CD81, CD63). Next, the membrane was cultured together with a secondary antibody conjugated to peroxidase. The membrane was treated with a chemiluminescence detection reagent (enhanced chemiluminescence), sensitized to an X-ray film, and developed to confirm the band.

### Preparation Example 7

Analysis of Exosomes Using Flow Cytometry (Fluorescence-Activated Cell Sorting; FACS)

Exosomes derived from the 5FU anticancer drug-resistant cell line (SNU-C5/5FUR) exposed to a normal oxygen or hypoxic environment were cultured together with CD81 or CD63 antibody (antibody for FACS with fluorescent material PE attached) for 30 minutes at room temperature, and then subjected to the measurement using a flow cytometer.

### Preparation Example 8

Invasion Assay Technique

The anticancer drug-resistant cell lines (SNU-C5/5FUR, SNU-C5/OXR) or endothelial progenitor cells (EPC) were treated with exosomes derived from anticancer drug-resistant cell lines (SNU-C5/5FUR, SNU-C5/OXR) exposed to a normal oxygen or hypoxic environment, and then spread on 8 μm pore insert culture plates coated with Matrigel (50 μl).

Thereafter, a growth medium was added to a low chamber plate, a serum-free medium was added to an insert culture plate, and culture was performed in the presence of 5% CO₂ in an incubator at 37° C. for 24 hours.

Next, the media were all removed, and the cells in the insert culture plate were fixed in 4% paraformaldehyde solution for 1 hour, stained with a crystal violet staining solution for 5 minutes, and imaged using an optical microscope.

### Preparation Example 9

Wound Healing Assay Technique

The vascular endothelial progenitor cells attached to the cell culture plate were removed using a cell scraper having a thickness of about 1 mm, and washed three times with PBS. The vascular endothelial progenitor cells were treated with exosomes derived from the 5FU anticancer drug-resistant cell line (SNU-C5/5FUR) exposed to a normal oxygen or hypoxic environment or exosomes derived from SNU-C5/5FUR treated with prion protein-targeting small interfering RNA (PRNP siRNA) or non-targeting siRNA (scrambled siRNA; si-Con), and then cultured in the presence of 5% CO₂ in an incubator at 37° C. for 24 hours. Thereafter, the distance narrowed from a distance of 1 mm after 24 hours was measured using an optical microscope.

### Preparation Example 10

Angiogenesis Antibody Array-Membrane Test

A membrane to which angiogenesis-related factor antibodies (total 43 targets) were attached was purchased (abcam; ab193655), and dot blot analysis was performed. The protein sample used was vascular endothelial progenitor cells treated with exosomes derived from the 5FU anticancer drug-resistant cell line (SNU-C5/5FUR) exposed to a normal oxygen or hypoxic environment or exosomes derived from SNU-C5/5FUR treated with prion protein-specific siRNA (si-PRNP) or non-targeting siRNA (si-Con). After being treated with exosomes, the vascular endothelial progenitor cells were cultured in the presence of 5% CO₂ in an incubator at 37° C. for 24 hours, and then the whole protein sample was obtained from the cells.

### Preparation Example 11

Rhodamine-Dextran Stain

Exosomes derived from the 5FU anticancer drug-resistant cell line (SNU-C5/5FUR) exposed to a normal oxygen or hypoxic environment (2 μg; twice a week, for a total of 2 weeks) were injected into the tail vein of mice, and Rhodamine-dextran (100 mg/kg) was injected into the tail vein of mice 3 hours before the biopsy. The liver and lung tissues of the mice were then biopsied and extracted. The extracted tissues were subjected to frozen tissue sectioning to prepare tissue slides, DAPI staining was performed, and then images were taken using a confocal fluorescence microscope.

### Preparation Example 12

Animal Model of Tumorigenesis

SNU-C5/WT (5×10⁶ cells), a general colorectal cancer cell line treated with exosomes derived from the 5FU anticancer drug-resistant cell line (SNU-C5/5FUR) exposed to a normal oxygen or hypoxic environment, was transplanted into the subcutaneous layer of mice. From a volume of 8 to 10 mm³ of tumors formed after 1 week, 5FU (30 mg/kg) and prion protein antibody were administered to the mice through the tail vein. The drugs were administered twice a week for a total of 4 weeks.

### Preparation Example 13

Animal Model of Tumor Metastasis

SNU-C5/WT (1×10⁷ cells), a general colorectal cancer cell line treated with exosomes derived from the 5FU anticancer drug-resistant cell line (SNU-C5/5FUR) exposed to a normal oxygen or hypoxic environment, was transplanted into the spleen of mice. After one week, 5FU (30 mg/kg) and prion protein antibody were administered to the mice through the tail vein. The drugs were administered twice a week for a total of 7 weeks. Thereafter, a biopsy was performed to confirm a tumor metastasized to the liver of the mice.

### Preparation Example 14

Immunofluorescence Staining

Tissues (tumor tissue, liver-metastasized tumor tissue) biopsied in the animal models of tumorigenesis and animal models of tumor metastasis were fixed in 4% paraformaldehyde, embedded in paraffin, and sectioned. Fluorescence staining was performed on the sectioned tissues using the cell proliferation-related factor Ki67, the apoptosis-related factor cleaved caspase-3, the cell-cell junction-related protein of vascular endothelial cells ZO-1, and the capillary marker CD31, and fluorescence staining was performed on the tissue nuclei using DAPI. Thereafter, images were taken using a confocal fluorescence microscope.

### Example 1

Confirmation of Prion Protein Expression in Plasma of Colorectal Cancer Patients and Three-Dimensional Sphere Forming Ability of Colorectal Cancer Cell Lines with Anticancer Drug Resistance

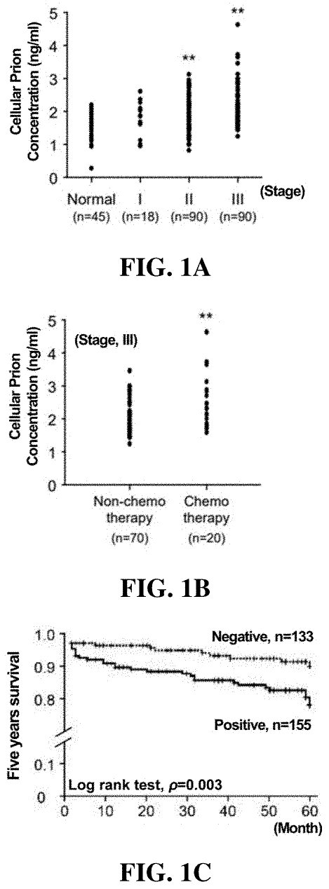

FIG. 1A illustrates colorectal cancer patients classified according to colorectal cancer progression, and the expression of prion protein according to each colorectal cancer progression stage (stage I, II, and III) measured by prion protein-specific enzyme-linked immunosorbent assay (ELISA). As a result, the expression of prion protein in the plasma of the patients increased as the cancer progression stage increased.

FIG. 1B illustrates patients in colorectal cancer progression stage 3 (stage III) classified according to the presence or absence of drug treatment, and the expression of prion protein in the plasma of the patients measured by ELISA. As a result, the expression of prion protein in the plasma of the patients with a history of drug treatment was high.

FIG. 1C illustrates colorectal cancer patients classified according to the presence or absence of prion protein expression in colorectal cancer tissues and the five-year survival rate of the patients measured. As a result, in the patients with increased prion protein expression in colorectal cancer tissues, the five-year survival rate was low.

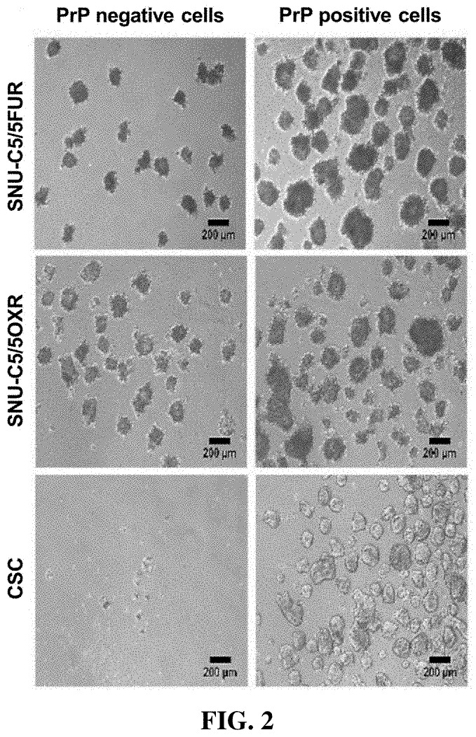

FIG. 2 illustrates cells isolated according to the presence or absence of prion protein expression in anticancer drug-resistant colorectal cancer cell lines (SNU-C5/5FUR, SNUC-C5/OXR) and a colorectal cancer stem cell line (CSC), and the tumor-forming ability of cancer cells confirmed through an experiment of forming three-dimensional spheres according to the presence or absence of prion protein expression. As a result, it was confirmed that sphere formation was promoted in colorectal cancer cells with high prion protein expression.

Consequently, the results of FIGS. 1A-1C and 2 suggest that the presence or absence of prion protein expression in colorectal cancer cells increases tumor-forming ability.

### Example 2

Identification of Exosomes Extracted from 5FU Anticancer Drug-Resistant Cell Lines and Prion Proteins Contained in Exosomes and their Effect on Colorectal Cancer Cells

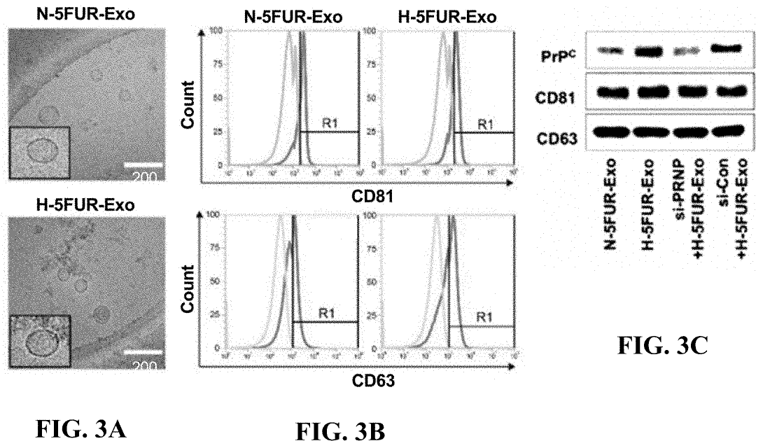

FIG. 3A is images obtained by extracting exosomes from a 5FU anticancer drug-resistant cell line and confirming whether the extracted exosomes are 200 μm or less using a cryogenic electron microscope.

FIG. 3B illustrates exosome markers CD81 and CD63 measured from extracted exosomes using a flow cytometry.

FIG. 3C illustrates prion protein expressed in extracted exosomes and exosome markers CD81 and CD63 confirmed by western blot analysis.

As a result, when the 5FU anticancer drug-resistant cell line was cultured in a hypoxic environment, the expression of prion protein in exosomes increased. However, as a result of suppressing the expression of prion protein in the 5FU anticancer drug-resistant cell line through PRNP siRNA, the expression of prion protein in exosomes did not increase even when the 5FU anticancer drug-resistant cell line was cultured in a hypoxic environment.

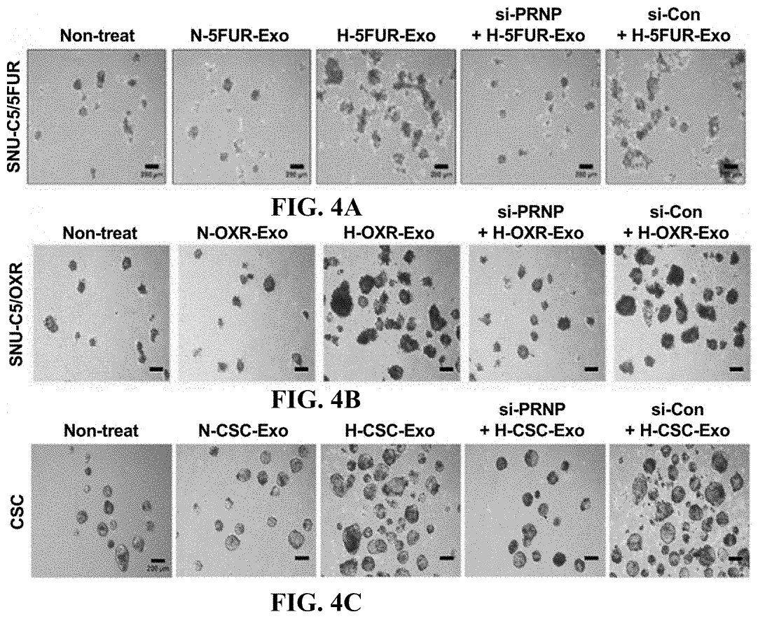

FIGS. 4A-4C illustrate the three-dimensional sphere-forming ability of colorectal cancer cell lines when anticancer drug-resistant colorectal cancer cell lines (SNU-C5/5FUR, SNU-C5/OXR) and a colorectal cancer stem cell line (CSC) exposed to a normal oxygen environment were treated with corresponding exosomes extracted from each of anticancer drug-resistant cell lines (SNU-C5/5FUR, SNU-C5/OXR) and a colorectal cancer stem cell line (CSC) cultured in a normal oxygen or hypoxic environment. FIG. 4A illustrates SNU-C5/5FUR that has been exposed to a normal oxygen environment and treated with exosomes extracted from SNU-C5/5FUR exposed to a normal oxygen or hypoxic environment, FIG. 4B illustrates SNU-C5/OXR that has been exposed to a normal oxygen environment and treated with exosomes extracted from SNU-C5/OXR exposed to a normal oxygen or hypoxic environment, and FIG. 4C illustrates CSC that has been exposed to a normal oxygen environment and treated with exosomes extracted from CSC exposed to a normal oxygen or hypoxic environment.

In all the groups treated with exosomes with high prion protein expression (H-5FUR-Exo, H-OXR-Exo and H-CSC-Exo), the three-dimensional sphere-forming ability, which was the tumor-forming ability, increased as compared to the groups treated with exosomes with low prion protein expression (N-5FUR-Exo, N-OXR-Exo and N-CSC-Exo). However, in the groups treated with exosomes without prion protein (si-PRNP+H-5FUR-Exo, si-PRNP+H-OXR-Exo and si-PRNP+H-CSC-Exo) extracted from anticancer drug-resistant colorectal cancer cell lines (SNU-C5/5FUR, SNU-C5/OXR) and a colorectal cancer stem cell line, which were treated with prion protein-targeting siRNA (si-PRNP) and cultured in a hypoxic environment, the three-dimensional sphere-forming ability was not improved. However, in the groups treated with exosomes (si-CON+H-5FUR-Exo, si-CON+H-OXR-Exo and si-CON+H-CSC-Exo) extracted from anticancer drug-resistant colorectal cancer cell lines (SNU-C5/5FUR, SNU-C5/OXR) and a colorectal cancer stem cell line, which were treated with non-targeting siRNA (si-Con) that was not able to inhibit any protein as a control of si-PRNP and cultured in a hypoxic environment, the three-dimensional sphere-forming ability, which was the tumor-forming ability, still increased.

### Example 3

Effect of Exosomes Extracted from Anticancer Drug-Resistant Cell Lines Cultured in Hypoxic Environment on Invasion Ability of Colorectal Cancer Cells

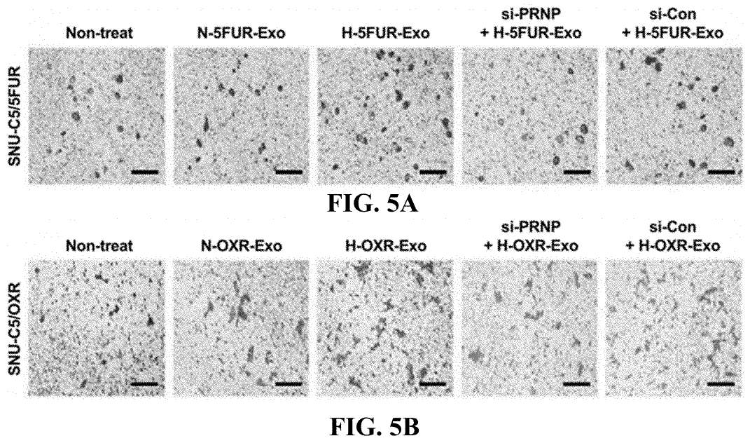

FIGS. 5A-5B illustrate the effect of exosomes extracted from anticancer drug-resistant cell lines cultured in a normal oxygen or hypoxic environment on the improvement of invasion ability of colorectal cancer cell lines (SNU-C5/5FUR, SNU-C5/OXR) confirmed by invasion assay.

As a result, in the groups treated with exosomes (H-5FUR-Exo, H-OXR-Exo) extracted from anticancer drug-resistant cell lines cultured in a hypoxic environment, the invasion ability of colorectal cancer cell lines increased. However, in the groups treated with the prion protein-inhibited exosomes (si-PRNP+H-5FUR-Exo, si-PRNP+H-OXR-Exo), the invasion ability of colorectal cancer cell lines was not improved.

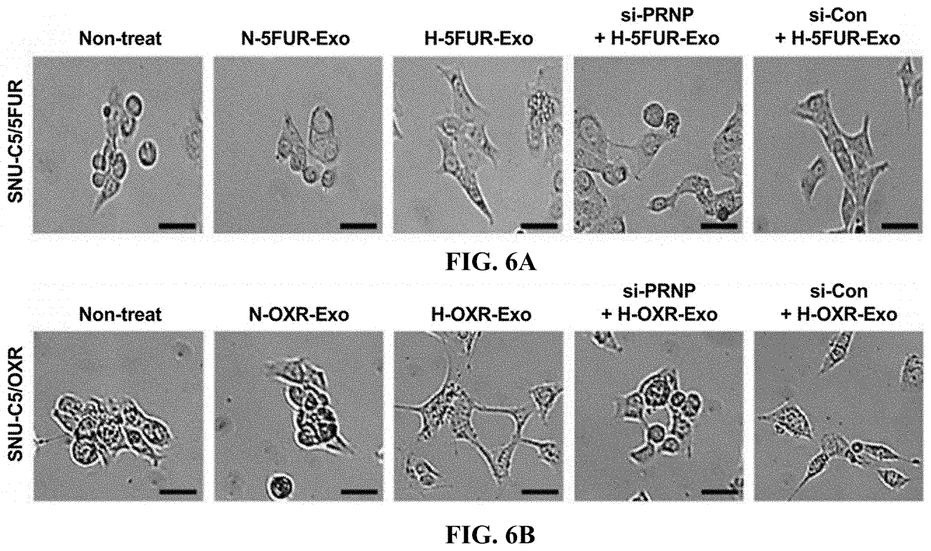

FIGS. 6A-6B illustrates images of the morphological changes of colorectal cancer cell lines treated with exosomes extracted from anticancer drug-resistant cell lines cultured in a normal oxygen or hypoxic environment taken using an optical microscope. Colorectal cancer cell lines treated with exosomes (H-5FUR-Exo, H-OXR-Exo) extracted from anticancer drug-resistant cell lines cultured in a hypoxic environment changed to a star or radial shape for cell migration. However, in the groups treated with the prion protein-inhibited exosomes (si-PRNP+H-5FUR-Exo, si-PRNP+H-OXR-Exo), there was no change in cell morphology for improvement of cell motility.

### Example 4

Effect of Exosomes Extracted from Anticancer Drug-Resistant Cell Lines Cultured in Hypoxic Environment on Vascular Endothelial Progenitor Cells

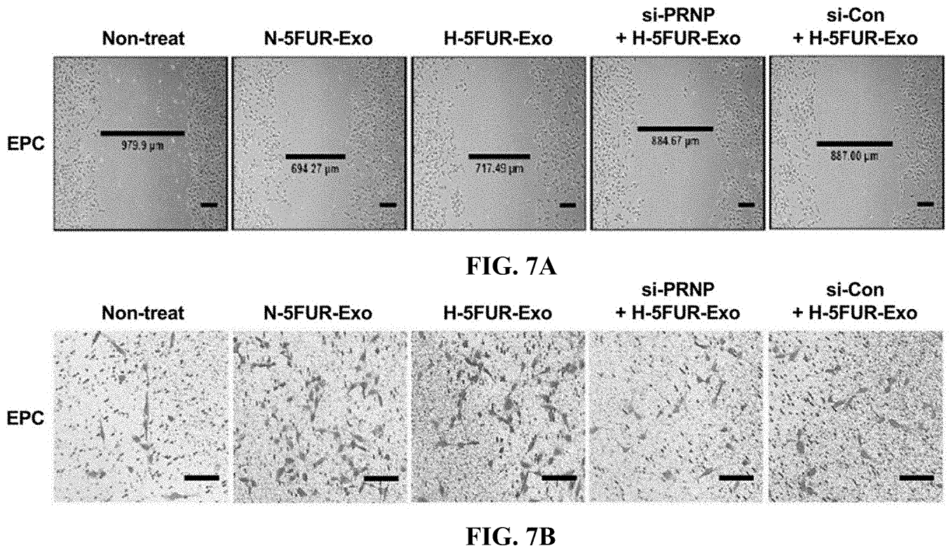

FIG. 7A illustrates the results of treating vascular endothelial progenitor cells treated with exosomes extracted from a 5FU anticancer drug-resistant cell line cultured in a normal oxygen or hypoxic environment and performing a cell migration assay to confirm migration ability of the cells.

FIG. 7B illustrates the results of treating vascular endothelial progenitor cells with exosomes extracted from a 5FU anticancer drug-resistant cell line cultured in a normal oxygen or hypoxic environment and performing an invasion assay to confirm migration ability of the cells.

As a result, the motility of the vascular endothelial progenitor cells was improved in the group treated with the exosomes (H-5FUR-Exo) extracted from a 5FU anticancer drug-resistant cell line cultured in a hypoxic environment. However, the motility of the vascular endothelial progenitor cells was not improved in the group treated with the prion protein-inhibited exosomes (si-PRNP+H-5FUR-Exo).

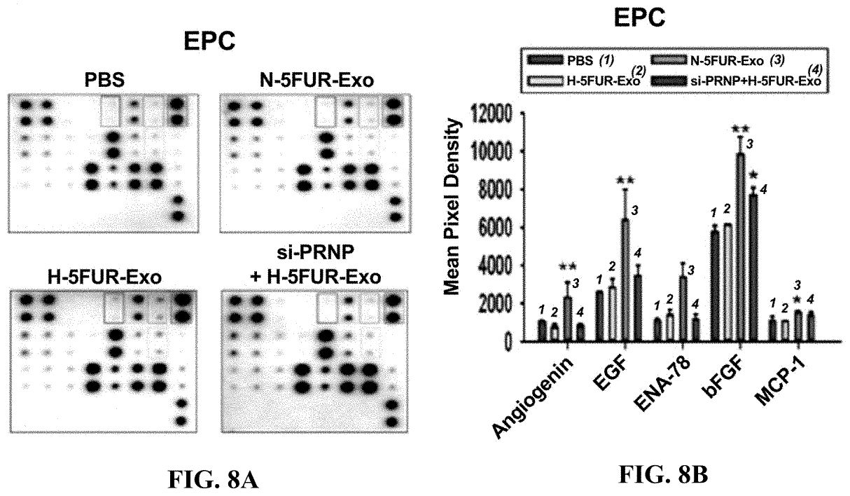

FIG. 8A illustrates the results of treating vascular endothelial progenitor cells with exosomes extracted from a 5FU anticancer drug-resistant cell line cultured in a hypoxic environment and measuring the expression level of angiogenic factors in the vascular endothelial progenitor cells by an Angiogenesis antibody array-membrane test.

FIG. 8B is a graph illustrating statistical values converted from the results of treating vascular endothelial progenitor cells with exosomes extracted from a 5FU anticancer drug-resistant cell line cultured in a hypoxic environment and measuring the expression level of angiogenic factors in the vascular endothelial progenitor cells by an Angiogenesis antibody array-membrane test.

As a result, the expression of angiogenic factors (Angiogenin, EGF, ENA-78, bFGF, MCP-1) increased in vascular endothelial progenitor cells treated with exosomes (H-5FUR-Exo) extracted from the 5FU anticancer drug-resistant cell line cultured in a hypoxic environment. However, the expression of angiogenic factors did not increase in vascular endothelial progenitor cells treated with prion protein-inhibited exosomes (si-PRNP+H-5FUR-Exo).

These results suggest that prion protein-positive exosomes secreted from colorectal cancer increase the motility of vascular endothelial cells that form blood vessels and promote the blood vessel formation.

### Example 5

Effect of Exosomes Extracted from Anticancer Drug-Resistant Cell Lines Cultured in Hypoxic Environment on Small Animal Experiments

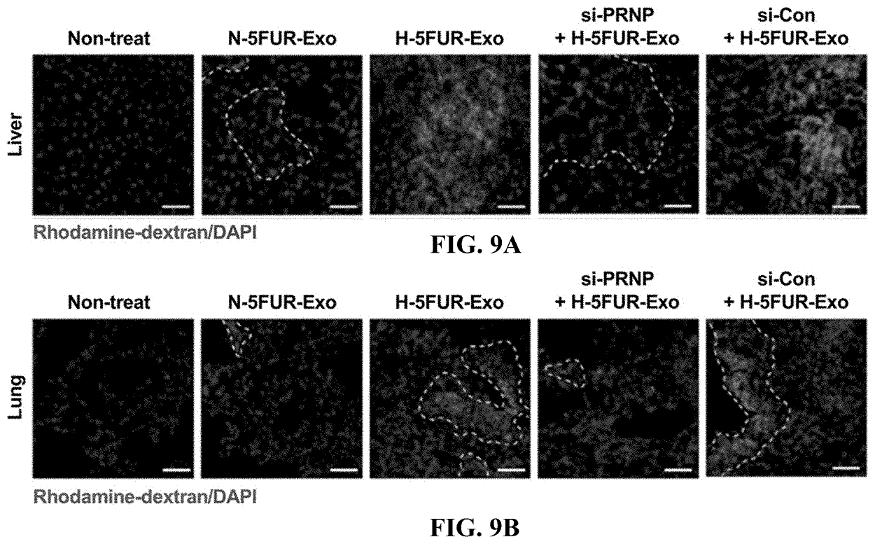

FIGS. 9A-9B illustrate the results of confirming the effect of exosomes on the tissue binding force of body organs when exosomes extracted from a 5FU anticancer drug-resistant cell line cultured in a normal oxygen or hypoxic environment are administered to experimental mice. The exosomes were administered twice a week for a total of 4 weeks, and then Rhodamine-dextran was administered to the mice through the tail vein 3 hours before the biopsy. Rhodamine-dextran has the characteristic of penetrating into areas where the tissue binding force of body organs is weakened. FIGS. 9A-9B have been confirmed by imaging using a fluorescence microscope. FIG. 9A is images of a mouse liver tissue, and FIG. 9B is images of a mouse lung tissue.

As a result, it was confirmed that the binding force of the liver and lung tissues of the experimental mouse administered with the exosomes (H-5FUR-Exo) extracted from an anticancer drug-resistant cell line cultured in a hypoxic environment was weakened so that Rhodamine-dextran penetrated into the tissues. These results suggest that exosomes extracted from an anticancer drug-resistant cell line cultured in a hypoxic environment weaken the tissue binding force of body organs. However, prion protein-inhibited exosomes (si-PRNP+H-5FUR-Exo) did not have any effect on the tissue binding force of body organs, and Rhodamine-dextran did not penetrate into the tissues of body organs.

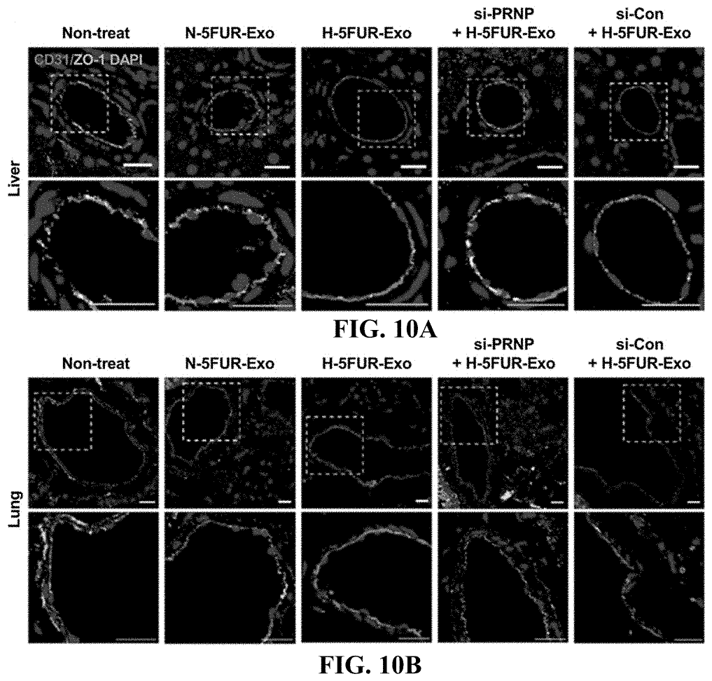

FIGS. 10A-10B illustrates the results of confirming the effect of exosomes on the cell-cell junction-related protein ZO-1 present in vascular tissues by fluorescence microscopy when exosomes extracted from a 5FU anticancer drug-resistant cell line cultured in a normal oxygen or hypoxic environment are administered to experimental mice. In FIGS. 10A-10B, fluorescence staining was performed on the blood vessels using CD31. FIG. 10A is an image of a mouse liver tissue, and FIG. 10B is an image of a mouse lung tissue.

As a result, it was confirmed that the exosomes (H-5FUR-Exo) extracted from an anticancer drug-resistant cell line cultured in a hypoxic environment decreased the expression of ZO-1 protein present in the vascular tissue. The decrease in expression of ZO-1 protein means that the cell-cell binding force of the tissue is decreased, and thus this result suggests that the colorectal cancer-derived exosomes weaken the cell-cell binding force of the vascular tissue present in the liver and lungs.

### Example 6

Confirmation of Inhibition of Colorectal Cancer Tumor Formation Using Combination of Prion Protein-Targeting Antibody and Anticancer Drug

A general colorectal cancer cell line group (PBS) without anticancer drug resistance, a group (Only 5-FU) in which a general colorectal cancer cell line without anticancer drug resistance was intraperitoneally administered with a 5FU anticancer drug (30 mg/kg) diluted with tertiary distilled water twice a week for a total of 7 weeks, a group (H-5FUR-Exo) in which a general colorectal cancer cell line without anticancer drug resistance was treated with exosomes extracted from a 5FU anticancer drug-resistant cell line cultured in a hypoxic environment, a group (H-5FUR-Exo+5-FU) in which a general colorectal cancer cell line without anticancer drug resistance was treated with exosomes extracted from a 5FU anticancer drug-resistant cell line cultured in a hypoxic environment and then intraperitoneally administered with 5FU anticancer drug (30 mg/kg) diluted with tertiary distilled water twice a week for a total of 7 weeks, a group (H-5FUR-Exo+5-FU+Anti-PrP) in which a general colorectal cancer cell line without anticancer drug resistance was treated with exosomes extracted from a 5FU anticancer drug-resistant cell line cultured in a hypoxic environment and then intraperitoneally administered with prion protein-targeting antibody (5 mg/kg) and 5FU anticancer drug (30 mg/kg) diluted with tertiary distilled water twice a week for a total of 7 weeks, and a group (H-5FUR-Exo+Anti-PrP) in which a general colorectal cancer cell line without anticancer drug resistance was treated with exosomes extracted from a 5FU anticancer drug-resistant cell line cultured in a hypoxic environment and then intraperitoneally administered with a prion protein-targeting antibody (5 mg/kg) diluted with tertiary distilled water twice a week for a total of 7 weeks were prepared, respectively.

The prion protein-targeting antibody used in Example 6 was the product number MAB1562 manufactured by Merck, United States.

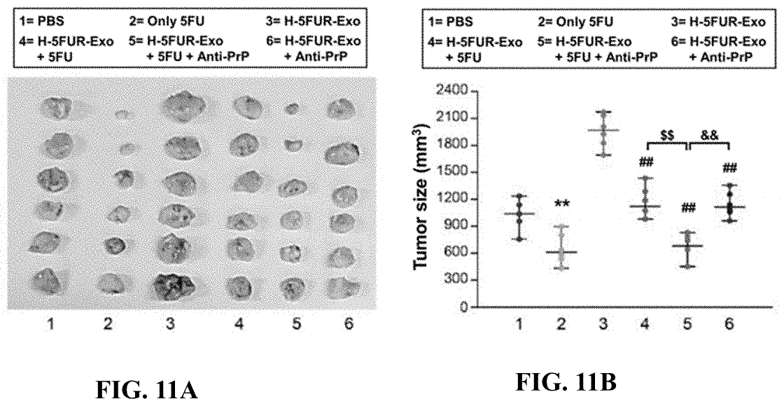

As a result, FIG. 11A is an image illustrating the state of formed tumors. FIG. 11B is a graph illustrating the volume distribution of formed tumors. According to these results, the general colorectal cancer cell line group (H-5FUR-Exo) treated with exosomes extracted from a 5FU anticancer drug-resistant cell line cultured in a hypoxic environment exhibited higher tumor-forming ability than the general colorectal cancer cell line (PBS) alone group.

The table shows that in the general colorectal cancer cell line group treated with exosomes extracted from a 5FU anticancer drug-resistant cell line cultured in a hypoxic environment, anticancer drug resistance is generated and treatment with 5FU anticancer drug alone (H-5FUR-Exo+5-FU) does not effectively inhibit tumor formation. However, in the group (H-5FUR-Exo+5-FU+Anti-PrP) in which a general colorectal cancer cell line group treated with exosomes extracted from a 5FU anticancer drug-resistant cell line cultured in a hypoxic environment was administered with a product prepared by diluting a prion protein-targeting antibody (5 mg/kg) and 5FU anticancer drug (30 mg/kg) with tertiary distilled water, tumor formation was effectively inhibited. These results are greatly similar to the results of the group (Only 5-FU) in which a general colorectal cancer cell line alone group without anticancer drug resistance is treated with 5FU anticancer drug, and it has been thus demonstrated that the combination of a prion protein-targeting antibody (5 mg/kg) and 5FU anticancer drug (30 mg/kg) prepared in the present invention increases the therapeutic effect in colorectal cancer with anticancer drug resistance.

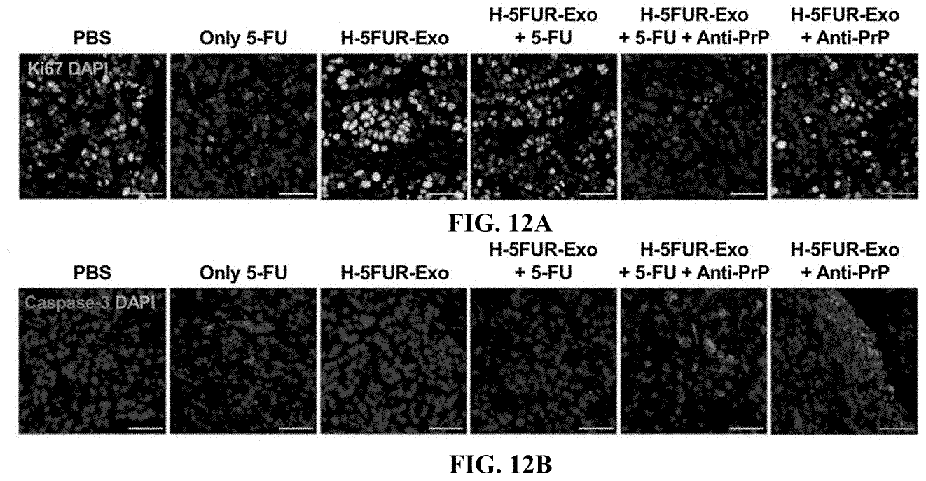

FIG. 12A illustrates the results of confirming cell proliferation of tumor cells through fluorescence staining of Ki67, a cell proliferation-related factor.

As a result, in the group (H-5FUR-Exo+5-FU) in which a general colorectal cancer cell line treated with exosomes extracted from a 5FU anticancer drug-resistant cell line cultured in a hypoxic environment was administered with 5FU anticancer drug (30 mg/kg), it was not able to decrease the proliferative ability of tumor cells even when 5FU anticancer drug was administered. However, in the group (H-5FUR-Exo+5-FU+Anti-PrP) in which a general colorectal cancer cell line group treated with exosomes derived from a 5FU anticancer drug-resistant cell line cultured in a hypoxic environment was administered with a product prepared by diluting a prion protein-targeting antibody (5 mg/kg) and 5FU anticancer drug (30 mg/kg) with tertiary distilled water, the cell proliferation rate of cancer cells in the tumor tissue significantly decreased.

FIG. 12B illustrates the results of confirming apoptosis through tissue fluorescence staining of cleaved caspase-3 used to confirm apoptosis and confocal fluorescence microscopy.

As a result, in the group (H-5FUR-Exo+5-FU+Anti-PrP) in which a general colorectal cancer cell line group treated with exosomes extracted from a 5FU anticancer drug-resistant cell line cultured in a hypoxic environment was administered with a product prepared by diluting a prion protein-targeting antibody (5 mg/kg) and 5FU anticancer drug (30 mg/kg) with tertiary distilled water, the expression of cleaved caspase-3 significantly increased.

These results are greatly similar to the results of the group (Only 5-FU) in which a general colorectal cancer cell line alone group without anticancer drug resistance is treated with 5FU anticancer drug, and it suggests that the combination of a prion protein-targeting antibody (5 mg/kg) and 5FU anticancer drug (30 mg/kg) prepared in the present invention promotes apoptosis of colorectal cancer cells with anticancer drug resistance.

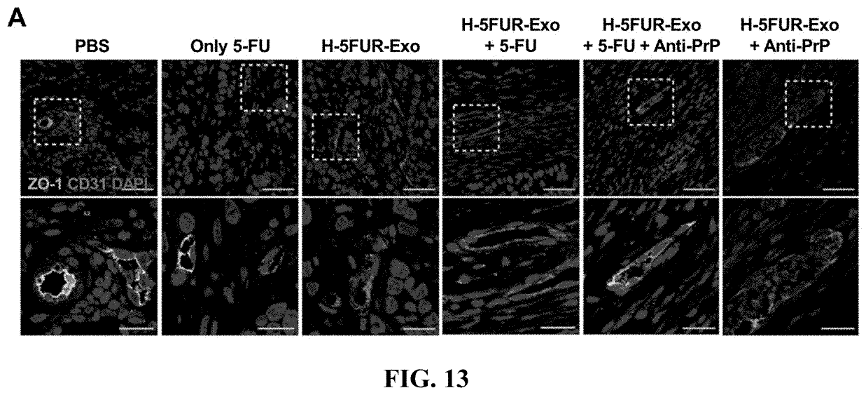

FIG. 13 illustrates the results of confirming through tissue fluorescence staining of blood vessels in the tumor tissue that the expression of cell-cell junction-related factor ZO-1 is maintained in the tumor tissue of an animal model of tumorigenesis in the group (H-5FUR-Exo+5-FU+Anti-PrP) in which a general colorectal cancer cell line group treated with exosomes extracted from a 5FU anticancer drug-resistant cell line cultured in a hypoxic environment was administered with a product prepared by diluting a prion protein-targeting antibody (5 mg/kg) and 5FU anticancer drug (30 mg/kg) with tertiary distilled water.

### Example 7

Effect of Combination of Prion Protein-Targeting Antibody and Anticancer Drug on Metastasis of Colorectal Cancer Tumor

The prion protein-targeting antibody used in Example 7 was the product number MAB1562 manufactured by Merck, United States.

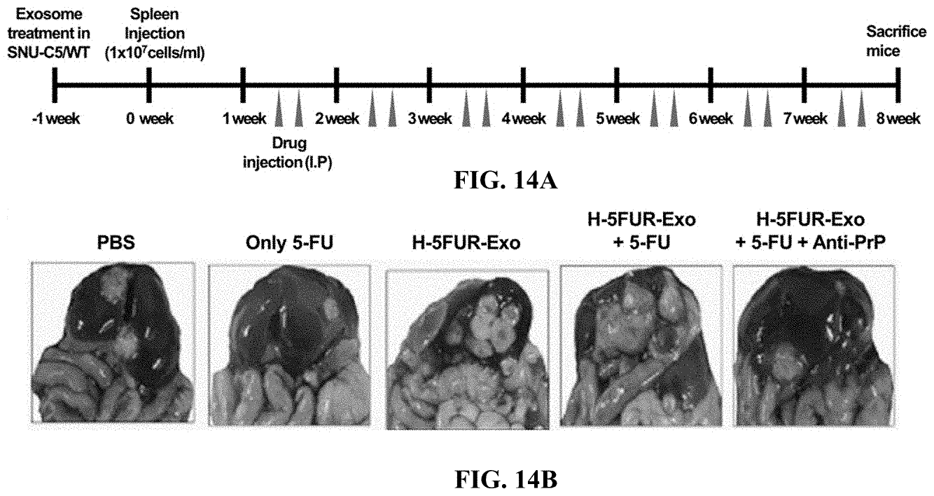

FIG. 14A is a schematic diagram illustrating a method of constructing an animal model of tumor metastasis by treating a general colorectal cancer cell line (SNU-C5/WT) without anticancer drug resistance with exosomes extracted from a 5FU anticancer drug-resistant cell line cultured in a hypoxic environment and then transplanting the general colorectal cancer cell line into the spleen of an experimental mouse, and after 1 week, intraperitoneally administering a prion protein-targeting antibody (5 mg/kg) and 5FU anticancer drug (30 mg/kg) diluted with tertiary distilled water twice a week for a total of 7 weeks.

FIG. 14B is an image illustrating metastasis of the cancer cells transplanted into the spleen of a mouse to the liver of the mouse.

As a result, in the general colorectal cancer cell line (H-5FUR-Exo) treated with exosomes derived from a 5FU anticancer drug-resistant cell line cultured in a hypoxic environment, metastasis to the liver was active. However, in the group (H-5FUR-Exo+5-FU+Anti-PrP) in which a general colorectal cancer cell line treated with exosomes derived from a 5FU anticancer drug-resistant cell line cultured in a hypoxic environment was administered with a combination of a prion protein-targeting antibody (5 mg/kg) and 5FU anticancer drug (30 mg/kg), the ability to metastasize to the liver decreased.

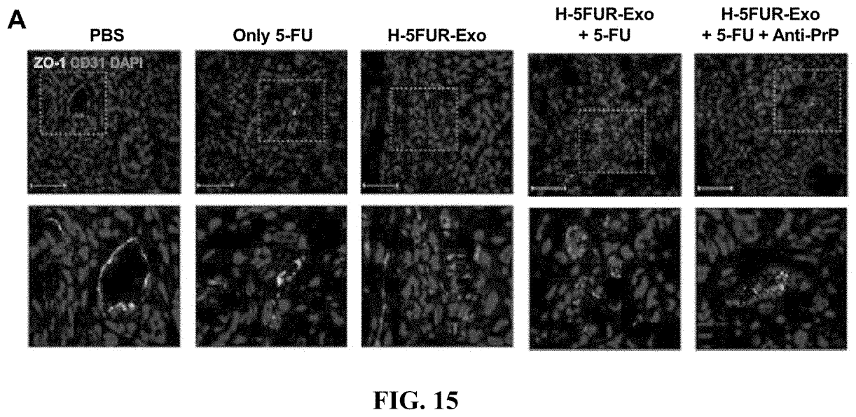

FIG. 15 illustrates the results of confirming through tissue fluorescence staining that the expression of cell-cell junction-related factor ZO-1 of vascular cells was maintained in the tumor tissues of a general colorectal cancer cell line treated with exosomes derived from a 5FU anticancer drug-resistant cell line cultured in a hypoxic environment metastasized to the liver of a small animal model of tumor metastasis in the group (H-5FUR-Exo+5-FU+Anti-PrP) in which a general colorectal cancer cell line treated with exosomes derived from a 5FU anticancer drug-resistant cell line cultured in a hypoxic environment was administered with a combination of a prion protein-targeting antibody (5 mg/kg) and 5FU anticancer drug (30 mg/kg).

Consequently, the present Example suggests that prion protein weakens the binding force of vascular cells so that the metastasis of colorectal cancer occurs actively and metastasis of colorectal cancer can be inhibited when the prion protein is inhibited.

### Example 8

Effect of Combination of Prion Protein-Targeting Antibody and Anticancer Drug on Cell Cycle of Anticancer Drug-Resistant Colorectal Cancer

The prion protein-targeting antibody used in Example 8 was the product number Sc-393165 manufactured by Santa Cruz Biotec, United States.

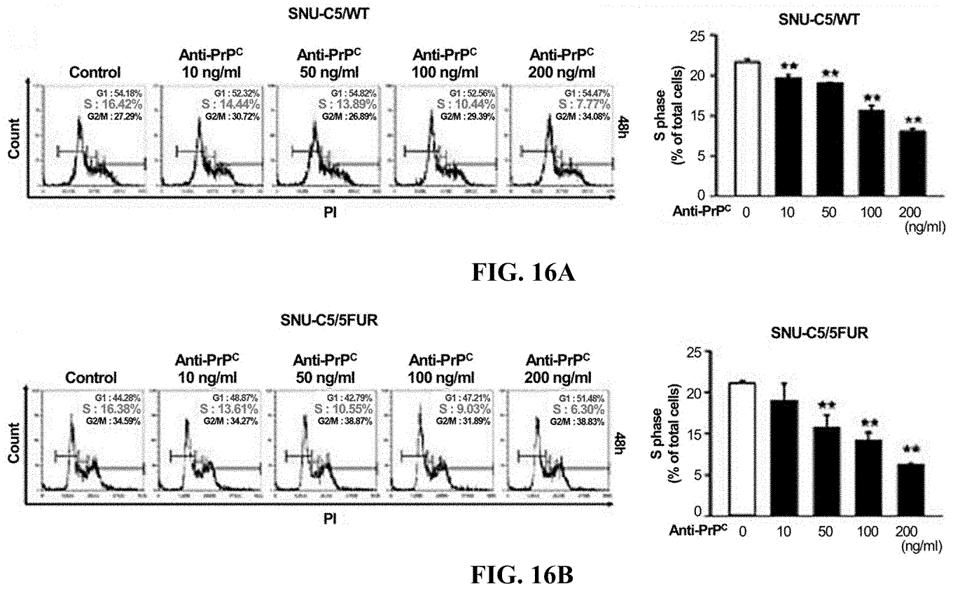

FIG. 16A illustrates the results of evaluating the proliferative ability of cells by cell cycle analysis when a general colorectal cancer cell line (SNU-C5/WT) without anticancer drug resistance is treated with a prion protein-targeting antibody at a concentration of 10 ng/ml, 50 ng/ml, 100 ng/ml, or 200 ng/ml.

FIG. 16B illustrates the results of evaluating the proliferative ability of cells by cell cycle analysis when a 5FU anticancer drug-resistant colorectal cancer cell line (SNU-C5/5FUR) is treated with a prion protein-targeting antibody at a concentration of 10 ng/ml, 50 ng/ml, 100 ng/ml, or 200 ng/ml.

As a result, it was confirmed that the cell cycle S phase of colorectal cancer cell line decreased as the concentration of prion protein-targeting antibody increased.

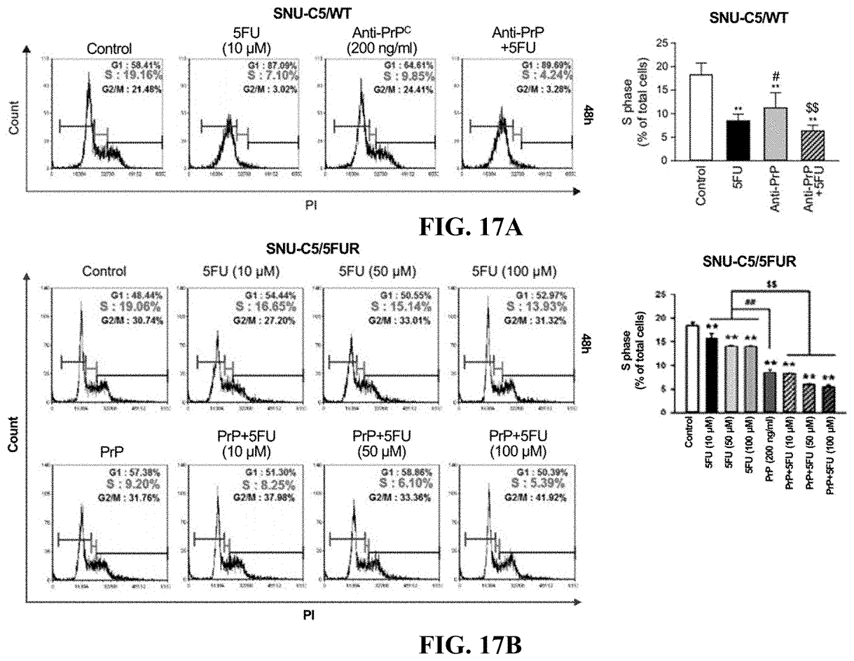

FIG. 17A illustrates the results of evaluating the proliferative ability of cells by cell cycle analysis when a general colorectal cancer cell line (SNU-C5/WT) without anticancer drug resistance is treated with 5FU anticancer drug (10 μM), a prion protein-targeting antibody (200 ng/ml), or 5FU anticancer drug and a prion protein-targeting antibody in combination.

FIG. 17B illustrates the results of evaluating the proliferative ability of cells by cell cycle analysis when a 5FU anticancer drug-resistant colorectal cancer cell line (SNU-C5/5FUR) is treated with 5FU anticancer drug (10, 50, 100 μM), a prion protein-targeting antibody (200 ng/ml), or 5FU anticancer drug (10, 50, 100 μM) and a prion protein-targeting antibody (200 ng/ml) in combination for each dose. FIG. 17B illustrates the results of confirming how high the anticancer drug sensitivity is by using 5FU anticancer drug at various concentrations since this 5FU anticancer drug-resistant colorectal cancer cell line is an anticancer drug-resistant cell.

As a result, the experimental group treated with a prion protein-targeting antibody and 5FU anticancer drug in combination inhibited the cell cycle S phase more than the group treated with 5FU anticancer drug alone. In particular, when the 5FU anticancer drug-resistant colorectal cancer cell line (SNU-C5/5FUR) is treated with 5FU anticancer drug at a concentration of 10, 50 or 100 μM at which anticancer drug resistance is exhibited and a prion protein-targeting antibody at the same time, it can be seen that the cell cycle S phase decreases up to one-third.

These results suggest that a prion protein-targeting antibody inhibits cell proliferation by enhancing the anticancer drug sensitivity to 5FU.

### Example 9

Effect of Combination of Prion Protein-Targeting Antibody and Anticancer Drug on Colorectal Cancer Formation

The prion protein-targeting antibody used in Example 9 was the product number Sc-393165 manufactured by Santa Cruz Biotec, United States.

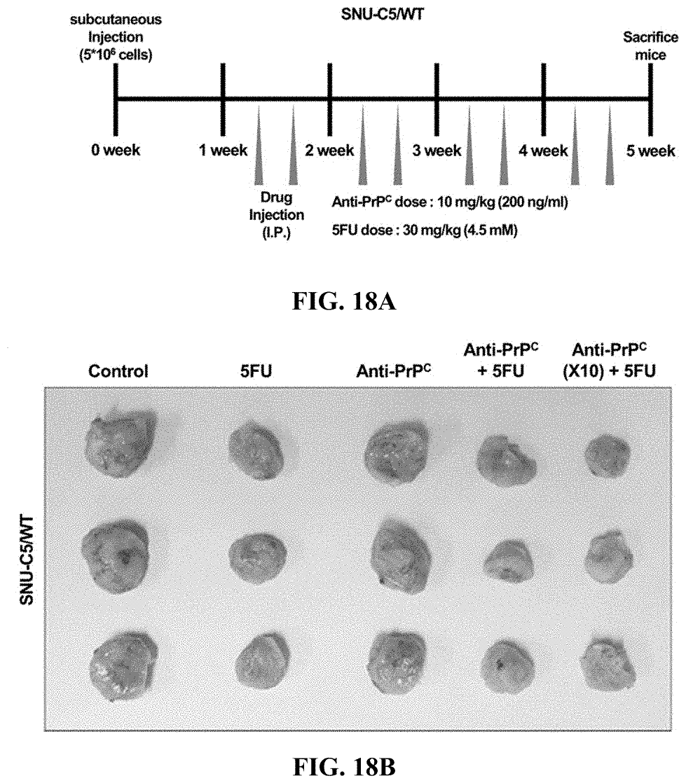

FIG. 18A is a schematic diagram illustrating an experimental method of constructing an animal model of tumorigenesis by transplanting a general colorectal cancer cell line (SNU-C5/WT) without anticancer drug resistance into a mouse and administering the animal model with 5FU anticancer drug and a prion protein-targeting antibody.

FIG. 18B is a result illustrating tumors formed when an animal model of tumorigenesis constructed by transplanting a general colorectal cancer cell line (SNU-C5/WT) without anticancer drug resistance into a mouse is treated with 5FU anticancer drug and a prion protein-targeting antibody in combination.

As a result, in the animal model group of tumorigenesis treated with 5FU anticancer drug and 10 mg/kg or 100 mg/kg of prion protein-targeting antibody in combination, the size of tumors decreased as compared to that in the group administered with 5FU anticancer drug alone. Consequently, it suggests that the sensitivity to an anticancer drug is enhanced and the anticancer therapeutic effect is enhanced when a prion protein-targeting antibody is used in a model of tumorigenesis.

As described above, although preferred embodiments of the present invention have been described with reference to the drawings, those of ordinary skill in the art will understand that various modifications and changes of the present invention can be made without departing from the spirit and scope of the present invention as set forth in the following claims.

### INDUSTRIAL APPLICABILITY

The pharmaceutical composition for cancer treatment comprising a prion protein-specific antibody of the present invention can enable the effect of an anticancer drug to act sensitively even in cancer cells that have developed resistance to the anticancer drug.

1. A pharmaceutical composition for cancer treatment comprising a prion protein-specific antibody.

2. The pharmaceutical composition for cancer treatment according to claim 1, which further comprises one or more selected from the group consisting of an anticancer drug, a chemotherapeutic agent, a toxin, a radioisotope, and a cytotoxic enzyme.

3. The pharmaceutical composition for cancer treatment according to claim 2, wherein the anticancer drug is one or more selected from the group consisting of 5FU anticancer drug and oxalaplatin anticancer drug.

4. A pharmaceutical composition for cancer treatment, comprising a complex of a target that binds to a prion protein antigenic determinant; and one or more selected from the group consisting of an anticancer drug, a chemotherapeutic agent, a toxin, a radioisotope, and a cytotoxic enzyme.
